(19) United States
(12) Patent Application Publication
(10) Pub. No.: US 2026/0047988 A1
(43) Pub. Date: Feb. 19, 2026

(54) HAIR COSMETIC COMPOSITIONS AND METHOD OF TREATING HAIR

(71) Applicant: L'OREAL, Paris (FR)

(72) Inventors: Gizele FREITAS, Rio de Janeiro (BR); Cyrus ANDRIOLO, Rio de Janeiro (BR); Érika Alegrio Jarque PETALI, Rio de Janeiro (BR); João Paulo Duarte GUIMARAES, Rio de Janeiro (BR)

(73) Assignee: L'OREAL, Paris (FR)

(21) Appl. No.: 19/101,644

(22) PCT Filed: Aug. 10, 2022
(86) PCT No.: PCT/BR2022/050313

§ 371 Date: Feb. 6, 2025

(51) Int. Cl. A61K 8/365; A61K 8/34; A61K 8/44; A61K 8/92; A61Q 5/12

(52) U.S. Cl. CPC A61K 8/365; A61K 8/345; A61K 8/44; A61K 8/922; A61Q 5/12

(57) ABSTRACT

The present invention is directed to hair cosmetic compositions, comprising at least one organic acid, a non-aqueous solvent comprising at least one compound with at least one hydroxyl-group, at least one amino acid, at least one fatty compound selected from vegetal oils, vegetal resins, vegetal waxes, and mixtures thereof, water, and at least one surfactant selected from cationic surfactants, nonionic surfactants, and mixtures thereof. The invention is also related to a method of treating hair, particularly curly hair.

### FIELD OF THE INVENTION

The present invention is directed to hair cosmetic compositions comprising (a) at least one organic acid, (b) a non-aqueous solvent comprising at least one compound with at least one hydroxyl-group, (c) at least one amino acid, (d) at least one at least one fatty compound selected from vegetal oils, vegetal resins, vegetal waxes, and mixtures thereof, (e) water, and (f) at least one surfactant selected from cationic surfactants, nonionic surfactants, and mixtures thereof. The invention is also related to a method of treating hair, particularly curly hair.

### BACKGROUND OF THE INVENTION

People have been using several cosmetic and personal care compositions to enhance the look and feel of keratin fibers, such as the hair. Particularly regarding curly hair, consumers are always dealing with problems related to the definition of the curls such as frizz, volume and shape. Curly hair tends to become frizzy and significantly expand in volume when exposed to high humidity conditions, chemical treatments, among others. Additionally, the shape and definition of the curls are lost after sleeping.

Many attempts to developed products to discipline, define and control the hair, in particular curly hair, enhancing/maintaining curl definition, curl retention, and curl regularity, without leaving the hair feeling and/or looking unnatural and stiff were made, but such products resulted in additional problems or maintenance difficulty.

For example, the products of the state of the art usually require high amount of product to give good combing and hair feel. But, considering that the effect of such products is not long lasting, the consumers have to reapply the product several times during the day or after sleeping in order to regain definition and frizz control, resulting in the build-up of the product on the hair's surface, resulting in a heavy aspect, wet look, unnatural touch and greasy sensation, since such products do not penetrate the keratin fibers and remain on the surface of the hair fiber.

In this sense, there is a need in the market for curly hair products that define the curls in a lasting way, without the necessity of reapplying the product until the next wash or after sleeping, also decreasing the need to use large amounts of the product between the washes.

Additionally, the standard curly hair routine includes the use of several cosmetic products through the entire length of the hair and must be reset every three days, since the curls tend to lose definition and fresh-looking aspect after such period—it is a very time-consuming routine that involves the consumers restarting all the steps in about every three days, including washing the hair and applying different cosmetic products.

Therefore, it is desirable to have a hair cosmetic product for curly hair that not only helps defining the curls, but also provide long lasting cosmetic benefits to the consumer's hair such as resistance to frizz, that leave the hair shiny and that is easy to apply and does not leave the hair feeling wet or heavy, that improve with the use of such product. There is also the need for the hair cosmetic product for curly hair to reduce the quantity of products used by the consumer, and to prolong the effects of the composition so the consumer may gain extra days before having to restart the curly hair cosmetic routine.

The inventors of the present invention surprisingly and unexpectedly achieved hair cosmetic compositions, particularly designed for curly hair, comprising (a) at least one organic acid, (b) a non-aqueous solvent comprising at least one compound with at least one hydroxyl-group, (c) at least one amino acid, (d) at least one fatty compound selected from vegetal oils, vegetal resins, vegetal waxes, and mixtures thereof, (e) water and (f) at least one surfactant selected from cationic surfactants, nonionic surfactants, and mixtures thereof, that are able to not only penetrate the hair fiber without building-up on the fiber's surface, while also providing curl definition until the next wash, long lasting of definition of the curls, frizz control, natural look and desirable volume, and that does not look heavy on the hair, but also provides cosmetic benefits such shine, gliding touch, spreadability. It was also discovered that these cosmetic benefits were more pronounced with the multi-application of the product (cumulative effect) and gives the consumer at least one more day before having to reset the curly hair routine.

### SUMMARY OF THE INVENTION

The present invention relates to hair cosmetic compositions comprising (a) at least one organic acid, (b) a non-aqueous solvent comprising at least one compound with at least one hydroxyl-group, (c) at least one amino acid, (d) at least one fatty compound selected from vegetal oils, vegetal resins, vegetal waxes, and mixtures thereof, (e) water, and (f) at least on surfactant selected from cationic surfactants, nonionic surfactants, and mixtures thereof. The present invention is also directed to a method for treating hair, particularly curly hair, comprising the step of contacting the hair with the hair cosmetic composition of the invention.

### DETAILED DESCRIPTION OF THE INVENTION

The present invention is directed to a hair cosmetic composition, mainly for curly hair, comprising:
(a) at least one organic acid;
(b) a non-aqueous solvent comprising at least one compound with at least one hydroxyl-group;
(c) at least one amino acid;
(d) at least one fatty compound selected from vegetal oils, vegetal resins, vegetal waxes, and mixtures thereof;
(e) water; and
(f) at least on surfactant selected from cationic surfactants, nonionic surfactants, and mixtures thereof.

In an embodiment, the at least one organic acid is selected from short chain organic acids, preferably α-hydroxy acids (AHAs), beta hydroxy acids, and a mixture thereof. In an embodiment, the at least one organic acid of the hair cosmetic composition of the present invention is selected from tartaric acid, lactic acid, glutaric acid, etidronic acid, salicylic acid, glycolic acid, citric acid, and mixtures thereof.

In a preferred embodiment, the at least one organic acid is tartaric acid.

In a preferred embodiment, the at least one organic acid is lactic acid.

In an embodiment, the at least one organic acid includes tartaric acid and a second organic acid selected from lactic acid, glutaric acid, etidronic acid, salicylic acid, glycolic acid, and citric acid.

In a preferred embodiment, the at least one organic acid includes tartaric acid and lactic acid.

Also, the amount of the at least one organic acid ranges from about 0.1% to about 25.0% by weight, 0.5% to about 20.0% by weight, preferably from about 0.5% to about 10.0% by weight, more preferably from about 0.75% to about 6.0% by weight, even more preferably from about 1.0% to about 5.0%, and most preferably from about 1.0% to about 4.0%, based on the total weight of the composition.

The hair cosmetic composition according to the present invention also comprises a non-aqueous solvent comprising at least one compound with at least one hydroxyl-group. Without being limited to any one theory, it is believed that the non-aqueous solvent comprising at least one compound with at least one hydroxyl-group acts as a penetrating agent, that is, being capable of forming the dipole-dipole interaction with the keratin fibers, such as hair, and binding to polar sites on the surface of said keratin fiber and decrease the shielding, thus promoting penetration of the hair cosmetic composition into the keratin fibers.

In an embodiment, the non-aqueous solvent comprising at least one compound with at least one hydroxyl-group of the hair cosmetic composition of the present invention is selected from polyols, C₂ to C₆ monoalcohols, and a mixture thereof.

In an embodiment, the non-aqueous solvent comprising at least one compound with at least one hydroxyl-group of the hair cosmetic composition of the present invention is selected from polyols. In an embodiment, the polyol is a glycol selected from propylene glycol, dipropylene glycol, butylene glycol, pentylene glycol, hexylene glycol, glycerin and mixtures thereof. In an embodiment, the polyol comprises propylene glycol and dipropylene glycol. In an embodiment, the polyol is glycerin. In an embodiment, the polyol comprises propylene glycol and glycerin.

In an embodiment, the non-aqueous solvent comprising at least one compound with at least one hydroxyl-group of the hair cosmetic composition of the present invention is selected from C₂ to C₆ monoalcohols. In an embodiment, the C₂ to C₆ monoalcohols include ethanol and/or propanol and/or isopropyl alcohol (isopropanol) and/or butanol and/or hexanol.

In an embodiment, the non-aqueous solvent comprising at least one compound with at least one hydroxyl-group of the hair cosmetic composition of the present invention comprises a mixture of polyols and C₂ to C₆ monoalcohols. In an embodiment, the non-aqueous solvent comprising at least one compound with at least one hydroxyl-group of the hair cosmetic composition of the present invention comprises propylene glycol and ethanol. In an embodiment, the non-aqueous solvent comprising at least one compound with at least one hydroxyl-group of the hair cosmetic composition of the present invention comprises propylene glycol and isopropyl alcohol. In an embodiment, the non-aqueous solvent comprising at least one compound with at least one hydroxyl-group of the hair cosmetic composition of the present invention comprises propylene glycol, isopropyl alcohol, and ethanol. In an embodiment, the non-aqueous solvent comprising at least one compound with at least one hydroxyl-group of the hair cosmetic composition of the present invention comprises propylene glycol, isopropyl alcohol, ethanol, and glycerin. In an embodiment, the non-aqueous solvent comprising at least one compound with at least one hydroxyl-group of the hair cosmetic composition of the present invention comprises propylene glycol, ethanol, and glycerin.

The amount of the non-aqueous solvent comprising at least one compound with at least one hydroxyl-group ranges from about 0.1% to about 20.0% by weight, preferably from about 0.3% to about 15.0% by weight, or from about 0.5% to about 12.0% by weight, or from about 0.5% to about 11.0% by weight, or from about 1.0% to about 11.0% by weight, based on the total weight of the composition.

According to the present invention, the at least one amino acid is selected 10 from arginine.

In an embodiment, the at least one amino acid is selected from arginine.

In an embodiment, the at least one amino acid is selected from glycine.

In an embodiment, the at least one amino acid is selected from arginine and glycine.

The amount of the at least one amino acid ranges from about 0.1% to about 7.0% by weight, preferably from about 0.1% to about 6.0% by weight, more preferably from about 0.1% to about 5.0% by weight, even more preferably from about 0.5% to about 3.0% weight, based on the total weight of the composition. Other suitable ranges are from about 0.5% to about 2.0% by weight, or from about 0.7% to about 1.2% by weight, based on the total weight of the composition.

In an embodiment, the at least one fatty compound selected from vegetal oils, vegetal resins, vegetal waxes, and mixtures thereof is selected from Copaiba Officinalis (Balsam Copaiba) resin (oil resin or resin form).

In a further embodiment, the at least one fatty compound selected from vegetal oils, vegetal resins, vegetal waxes, and mixtures thereof is selected from coconut oil, Soja Soybean oil (soybean oil) and mixtures thereof.

In an embodiment, the at least one fatty compound includes coconut oil.

In an embodiment, the at least one fatty compound includes Soja Soybean oil.

In an embodiment, the at least one fatty compound selected from vegetal oils, vegetal resins, vegetal waxes, and mixtures thereof includes Copaiba Resin, Candeia resin, and white breu resin.

In an embodiment, the at least one fatty compound selected from vegetal oils, vegetal resins, vegetal waxes, and mixtures thereof is copaiba resin.

In an embodiment, the at least one fatty compound selected from vegetal oils, vegetal resins, vegetal waxes, and mixtures thereof includes copaiba resin and coconut oil, and Soja Soybean oil.

In an embodiment, the at least one fatty compound selected from vegetal oils, vegetal resins, vegetal waxes, and mixtures thereof includes copaiba resin and coconut oil.

In an embodiment, the at least one fatty compound selected from vegetal oils, vegetal resins, vegetal waxes, and mixtures thereof includes Candelilla wax.

In another embodiment, the at least one fatty compound selected from vegetal oils, vegetal resins, vegetal waxes, and mixtures thereof includes Carnauba wax.

The amount of the at least one fatty compound selected from vegetal oils, vegetal resins, vegetal waxes, and mixtures thereof ranges from about 0.1% to about 15.0% by weight, preferably from about 0.1% to about 14.0% by weight, preferably from about 0.15% to about 10.0% by weight, preferably from about 0.17% to about 4.0% by weight, or such as at about 0.1%, about 0.17%, about 0.2%, about 0.3%, about 0.36%, about 0.4%, about 0.5% to about 4.0% by weight, based on the total weight of the composition.

In an embodiment, the at least one surfactant selected from cationic surfactants, nonionic surfactants, and mixtures thereof includes at least one cationic surfactant. In a preferred embodiment, the at least one cationic surfactant is selected from quaternary ammonium salts such as cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, and from cationizable surfactants, for example, amido amine compounds such as oleamidopropyl dimethylamine, linoleamidopropyl dimethylamine, isostearamidopropyl dimethylamine, stearamidopropyl dimethylamine, behenamidopropyl dimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, and mixtures thereof.

In an embodiment, the at least one surfactant selected from cationic surfactants, nonionic surfactants, and mixtures thereof includes at least one cationic surfactant selected from behetrimonium chloride, cetrimonium chloride, behentrimonium methosulfate, and mixtures thereof.

In an embodiment, the at least one surfactant selected from cationic surfactants, nonionic surfactants, and mixtures thereof includes at least one cationic surfactant selected from stearamidopropyl dimethylamine, brassicamidopropyldimethylamine, and mixtures thereof.

In an embodiment, the at least one surfactant selected from cationic surfactants, nonionic surfactants, and mixtures thereof includes at least one nonionic surfactant. In a preferred embodiment, the at least one nonionic surfactant is selected from fatty alcohols.

In an embodiment, the fatty alcohols include cetyl alcohol, cetearyl alcohol, stearyl alcohol, lauryl alcohol, myristyl alcohol, and mixtures thereof.

In an embodiment, the fatty alcohols include cetearyl alcohol.

In an embodiment, the fatty alcohols include cetyl alcohol.

In an embodiment, the fatty alcohols include stearyl alcohol.

In an embodiment, the fatty alcohols include cetearyl alcohol and stearyl alcohol.

In an embodiment, the fatty alcohols include an alkoxylated alcohol.

In an embodiment, the fatty alcohols include an alkoxylated alcohol selected from steareth-20, Laureth-12, ceteareth-20, deceth-10, trideceth-6, and mixtures thereof.

In an embodiment, the at least one surfactant includes at least one cationic surfactant selected from behetrimonium chloride, cetrimonium chloride, behentrimonium methosulfate, stearamidopropyl dimethylamine, brassicamidopropyldimethylamine, and mixtures thereof, and at least one nonionic surfactant selected from cetyl alcohol, cetearyl alcohol, stearyl alcohol, lauryl alcohol, myristyl alcohol, and mixtures thereof.

When the at least one surfactant includes an amido amine such as stearamidopropyl dimethylamine or brassicamidopropyldimethylamine, a pH adjuster agent such as triethanolamine may be included in the composition.

The amount of the at least one surfactant selected from cationic surfactants, nonionic surfactants, and mixtures thereof ranges from about 0.05% to about 15.0% by weight, preferably from about 1.0% to about 14.0% by weight, preferably from about 1.5% to about 12.0% by weight, more preferably from about 1% to about 12% by weight, from about 2.0% to about 11.0%, from about 3.0% to about 10.0% by weight, including all ranges and subranges, based on the total weight of the hair cosmetic composition.

Individually, each surfactant may be present in an amount such as about 0.05%, about 0.5%, about 1.0%, about 1.5%, about 1.8%, about 2.4%, about 3.0%, about 3.5%, about 4.0%, about 5.0%, about 5.5%, about 6.0%, about 6.5%, about 7.0, about 7.5%, about 8.0%, about 8.5%, about 9.0%, about 9.5%, about 10. 0%, about 10.5%, about 11.0%, about 11.5%, about 12.0% by weight, based on the total weight of the composition.

In a preferred embodiment, the at least one surfactant is at least one cationic surfactant ranging from about 0.1% to about 10.0% by weight, such as from about 1.0% to about 10.0% by weight, from about 1.4% to about 9.0% by weight, from about 1.4% to about 8.0% by weight, from about 1.4% to about 6.0% by weight, from about 2.0 to about 6.0% by weight, from about 2.4 to about 6.0% by weight, based on the total weight of the composition.

In a preferred embodiment, the at least one surfactant is at least one nonionic surfactant selected from at least one fatty alcohol ranging from about 0.1 to about 12.0% by weight, such as from about 3.0 to about 12.0% by weight, from about 4.0% to about 12.0% by weight, from about 5.0% to about 10.0% by weight, from about 6.0% to about 9.0% by weight based on the total weight of the composition.

In an embodiment, the hair cosmetic composition of the present invention further comprises at least one ester, which may be selected from isopropyl palmitate, dicaprylyl carbonate, cetyl esters, and mixtures thereof.

In a preferred embodiment, the hair cosmetic composition of the present invention is in the form of an emulsion (oil in water or water in oil).

In a further preferred embodiment, the hair cosmetic composition can be, but not exclusive, free of or substantially free of silicon, behentrimonium chloride, and/or a quaternary ammonium salt.

In an embodiment, the hair cosmetic compositions according to the invention are free of or substantially free of anionic surfactants.

The pH of the hair cosmetic composition of the present invention is from about 1 to about 10, preferably from about 2 to about 7, or from about 2 to about 6. To achieve lasting of definition, the pH is about 3 to about 5, preferably from about 2.5 to about 4.5, or from about 3.0 to about 4.5, or from about 3.5 to about 4.5, or from about 3.0 to about 4.0, or at about 2, about, 3, about 3.3, about 3.5, about 4, about 4.5.

Moreover, the present invention is directed to a method for treating hair, particularly curly hair, comprising the step of contacting the hair with the hair cosmetic composition of the invention.

In a preferred embodiment, the composition of the invention is used as a leave-on or rinse-off product to deliver long lasting definition of curls without build up and frizz, that improve curl regularity, discipline, volume control, individualization of the curls, and dries fast, among other attributes.

### Organic Acid

The at least one organic acid of the compositions can be selected from alpha hydroxy acids, beta hydroxy acids, and a mixture thereof.

The term “alpha-hydroxy acid” is understood to mean, according to the present invention, a carboxylic acid having at least one hydroxyl functional group occupying an alpha-position on said acid (carbon adjacent to a carboxylic acid functional group). A beta-hydroxy acid is closely related to an alpha-hydroxy acid except the carboxylic and hydroxyl functional groups are separated by two carbon atoms. A hydroxy acid can be present in the final composition in the form of the free acid and/or in the form of one of its associated salts (salts with an organic base or an alkali metal, in particular), especially according to the final pH imposed on the composition.

The alpha hydroxy acid present in the composition, according to the disclosure, includes, but is not limited to, one or more of tartaric acid, glycolic acid, citric acid, lactic acid, methyllactic acid, glucuronic acid, pyruvic acid, 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytetradecanoic acid, 2-hydroxyhexadecanoic acid, 2-hydroxyoctadecanoic acid, 2-hydroxytetracosanoic acid, 2-hydroxyeicosanoic acid, mandelic acid, phenyllactic acid, gluconic acid, galacturonic acid, aleuritic acid, ribonic acid, tartronic acid, malic acid, fumaric acid, their salts and their mixtures. It is also possible to use mixtures of these various acids.

In some embodiments, the composition includes alpha hydroxy acids comprising tartaric acid and lactic acid. When combined, the more than one alpha hydroxy acids may be present in the same or in different amounts.

The beta hydroxy acid, when present in the composition according to the disclosure, includes, but is not limited to, one or more of salicylic acid and derivatives thereof (including 5-n-octanoylsalicylic acid, salicylate, sodium salicylate, and willow extract), beta hydroxybutanoic acid, tropic acid, and trethocanic acid.

In some embodiments, the composition includes tartaric acid.

In some embodiments, the composition includes tartaric acid and lactic acid.

Compound with at Least One Hydroxyl-Group

The non-aqueous solvent of the present invention comprises at least one compound with at least one hydroxyl-group.

Said compound with at least one hydroxyl-group can be selected from polyols, and/or C₂ to C₆ monoalcohols such as ethyl alcohol, isopropyl alcohol, propyl alcohol, ethylene glycol, propylene glycol, butylene glycol, hexylene glycol, propane diol, and glycerin.

Further non-limiting examples include alkanediols (polyhydric alcohols) such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, isopropanol, and a mixture thereof.

In some cases, the compound with at least one hydroxyl-group may be selected from the group consisting of one or more glycols, C1-4 alcohols, glycerin, and a mixture thereof. In some cases, the compound with at least one hydroxyl-group is selected from the group consisting of hexylene glycol, propylene glycol, dipropylene glycol, caprylyl glycol, glycerin, isopropyl alcohol, and a mixture thereof.

Polyhydric alcohols are useful. Examples of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof.

Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof.

### Amino Acid

The compositions according to the disclosure comprise at least one amino acid. As used herein, the term “amino acid” includes amino acids such as proteinogenic amino acids, amino sulfonic acids, and salts thereof.

Amino acids are simple organic compounds containing both a carboxylic acid group (—COOH) and an amino group (—NH₂). Amino sulfonic acids are simple organic compounds containing both a sulfonic acid group (—SO₂OH) and an amino group (—NH₂). Accordingly, the amino acids useful according to the disclosure may, in certain embodiments, be selected from compounds of Formula (I) and Formula (II):

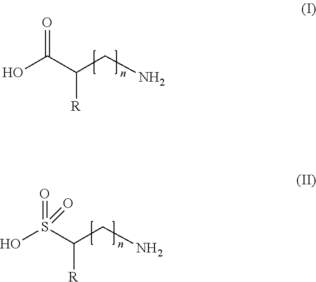

wherein:
   R represents a hydrogen atom, a linear or branched, preferably linear, C₁-C₅ alkyl group, said alkyl group being optionally substituted with at least one group chosen from hydroxyl, —C(O)—OH, —S(O)₂—OH, —C(O)—O— and M⁺, and S(O)₂—O— and M⁺, with M⁺ representing a cationic counter-ion such as an alkali metal, alkaline earth metal, or ammonium, and
   n is 0 or 1.

The amino acids may be in their non-ionized form (I) and (II) or in their ionized or betaine form (I′) and (II′):

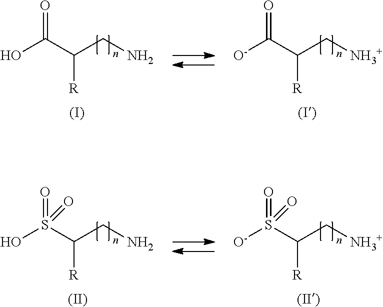

wherein R and n are as defined above.

The one or more amino acids may also be in their conjugate base form (Ib) and (IIb):

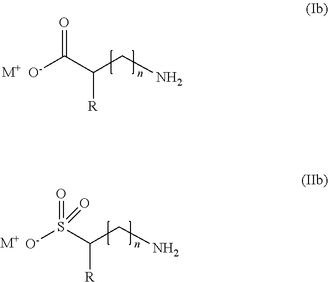

wherein R and n are as defined above.

Well-known amino acids include the twenty amino acids that form the proteins of living organisms (standard proteinogenic amino acids): alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. The amino acids of the instant disclosure, however, are not limited to the standard proteinogenic amino acids.

Non-limiting examples of amino sulfonic acids include aminomethane sulfonic acid, 2-aminoethane sulfonic acid (taurine), aminopropane sulfonic acid, aminobutane sulfonic acid, aminohexane sulfonic acid, aminoisopropyl sulfonic acid, aminododecyl sulfonic acid, aminobenzene sulfonic acid, aminotoulene sulfonic acid, sulfanilic acid, chlorosulfanilic acid, diamino benzene sulfonic acid, amino phenol sulfonic acid, amino propyl benzene sulfonic acid, amino hexyl benzene sulfonic acid, and a mixture thereof.

In some cases, charged amino acids may be used. Non-limiting examples of charged amino acids include arginine, lysine, aspartic acid, and glutamic acid. In some cases, polar amino acids are useful. Non-limiting examples of polar amino acids include glutamine, asparagine, histidine, serine, threonine, tyrosine, cysteine, methionine, and tryptophan.

In some cases, hydrophobic amino acids may be employed. Non-limiting examples of hydrophobic amino acids include alanine, isoleucine, leucine, phenylalanine, valine, proline, and glycine.

### Fatty Compound

The at least one fatty compound of the hair cosmetic composition of the present invention may be selected from vegetal oils, vegetal resins, vegetal waxes, and mixtures thereof.

### Vegetal Oil

In an embodiment, the fatty compound of the hair cosmetic composition of the present invention selected from vegetal oils, vegetal resins, vegetal waxes, and mixtures thereof comprises at least one vegetal oil.

In a non-limitative and preferred embodiment, the at least one vegetal oil is selected from coconut oil, almond oil, aloe vera oil, avocado oil, soybean oil, camelina oil, coffee oil, castor oil, grapeseed oil, jojoba oil, basil oil, bay oil, bergamot oil, black pepper essential oil, birch oil, camphor, cannabis oil, caraway oil, cardamom seed oil, carrot seed oil, chamomile oil, calamus root oil, cinnamon oil, citronella oil, clary sage, clove leaf oil, coffee, coriander, costmary oil, cranberry seed oil, cubeb, cumin oil, dill oil, eucalyptus oil, fennel seed oil, fenugreek oil, fir, frankincense oil, geranium oil, ginger oil, grapefruit oil, grapeseed oil, henna oil, horseradish oil, jasmine oil, juniper berry oil, lavender oil, lemon oil, lemongrass, marjoram, melaleuca, lemon balm oil, mountain savory, mugwort oil, mustard oil, myrrh oil, myrtle, neem tree oil, neroli, nutmeg, orange oil, oregano oil, orris oil, palo santo, parsley oil, peppermint oil, pine oil, plum oil, rose oil, rosehip oil, rosemary oil, rosewood oil, sandalwood oil, spruce, star anise oil, tarragon oil, tea tree oil, thyme oil, and mixtures thereof.

In some embodiments, the suitable vegetal oils for use in the hair care compositions of the present invention are selected from coconut oil, soybean oil, canola oil, rapeseed oil, corn oil, cottonseed oil, olive oil, palm oil, peanut oil, safflower oil, sesame oil, sunflower oil, linseed oil, palm kernel oil, wheatgerm oil, apricot kernel oil, pistachio oil, poppy oil, hazel nut oil, colza oil, cade oil, peach kernel oil, jojoba oil, Helianthus annuus (sunflower) seed oil, and mixtures thereof.

When present, the vegetal oil can improve the penetration effect.

In a preferred embodiment, the vegetal oil of the present invention is coconut oil, that is mainly composed by Lauric acid, Myristic Acid, Palmitic Acid, Oleic Acid, Caprylic Acid, Capric Acid. Other oils with the same composition could apply on the invention, such as Babassu oil.

In another preferred embodiment, the hair cosmetic composition of the present invention further comprises at least one vegetal oil selected from, argan oil, soybean oil and mixtures thereof. In a preferred embodiment, the hair cosmetic compositions of the present invention comprise a mixture of vegetal oils and vegetal resins, such as coconut oil and Copaiba Officinalis (Balsam Copaiba resin) (copaiba resin or copaiba oleoresin).

### Vegetal Resin

In an embodiment, the fatty compound selected from vegetal oils, vegetal resins, vegetal waxes, and mixtures thereof of the hair cosmetic composition of the present invention comprises at least one vegetal resin.

Preferably, the vegetal resin of the present invention is Copaifera officinalis (BALSAM COPAIBA) RESIN (INCI name) which can be in the form of a Copaiba oil resin or Copaiba resin. Copaifera officinalis (BALSAM COPAIBA) RESIN is commercially available under the tradename of RAIN FOREST RF3310 (also known by the chemical name of Copaiba Oleoresin) or under the tradename of RAIN FOREST 03370 (also known by the chemical name of Copaiba resin), both from the supplier Beraca Ingredientes Naturais.

### Vegetal Waxes

In an embodiment, the fatty compound selected from vegetal oils, vegetal resins, vegetal waxes, and mixtures thereof comprises a vegetal wax.

When present, the vegetal wax may be selected from Candelilla wax, Carnauba wax, beeswax, and mixtures thereof.

### Water

The hair cosmetic compositions according to the present invention comprise water. The water used may be sterile demineralized water and/or deionized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-bains, water from Saint Gervais-les-Bains, water from Neris-les-Bains, water from Allevar-les-Bains, water from Digne, water from Maizieres, water from Neyrac-les-Bains, water from Lons-le-saunier, water from EauxBonnes, water from Rochefort, water from Saint Christau, water from Les Fumades, water from Tercis-les-Bains or water from Avene. Water may also comprise reconstituted thermal water, that is to say a water comprising trace elements such as zinc, copper, magnesium, etc., reconstituting the characteristics of a thermal water.

The hair cosmetic composition according to the present invention may comprise water in an amount ranging from about 30.0% to about 99.0% by weight, preferably from about 65.0% to about 95.0% by weight, and most preferably from about 70.0% to about 92.0% by weight, relative to the total weight of the hair cosmetic composition.

### Esters

In preferential embodiments, the hair cosmetic composition of the present invention comprises at least one ester. The at least one ester may be selected from, but not limited to, isopropyl palmitate, dicaprylyl carbonate, cetyl esters, and mixtures thereof.

When present, the amount of the at least one ester ranges from about 0.5% to about 10.0% by weight, from about 1.5% to about 5.0% by weight, from about 0.5% to about 2.5% by weight, from about 1.0% to about 3.0% by weight, from about 2.0% to about 5.0% by weight, from about 2.0% to about 3.0% by weight, from about 5.0% to about 8.5% by weight, including all ranges and subranges, based on the total weight of the composition.

### Additional Ingredients

In addition to the essential components described hereinbefore, the hair composition of the present invention may further comprise any usual cosmetically acceptable ingredient, which may be chosen especially from such as cosmetically acceptable ingredients selected from perfume/fragrance, preserving agents, vitamins, vegetal extracts, pH-adjusters, and mixtures thereof.

A person skilled in the art will take care to select the optional additional ingredients and/or the amount thereof such that the advantageous properties of the composition according to the invention are not, or are not substantially, adversely affected by the envisaged addition.

The additional ingredients may represent from about 1% to about 70% by weight, based on the total weight of the composition.

### Terms

As used herein, the expression “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within +/−5% of the indicated number.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The term “substantially free” or “essentially free” as used herein means that there is less than about 5% by weight of a specific material added to a composition, based on the total weight of the compositions. Nonetheless, the compositions may include less than about 2 wt. %, less than about 1 wt. %, less than about 0.5 wt. %, less than about 0.1 wt. %, less than 0.01 wt. %, or none of the specified material.

The term “active material” as used herein with respect to the percent amount of an ingredient or raw material, refers to 100% activity of the ingredient or raw material.

“Cosmetically acceptable” means that the item in question is compatible with a keratinous substrate such as skin and hair. For example, a “cosmetically acceptable carrier” means a carrier that is compatible with a keratinous substrate such as skin and hair.

The term “hair cosmetic composition” as used herein can also be a hair styling or a hair shaping or a hair care or a hair treatment composition.

The term “cationic surfactant” means a surfactant that is positively charged. This surfactant may bear one or more positive permanent charges or may contain one or more functions that are cationizable in the composition according to the disclosure

Throughout the disclosure, the term “a combination thereof” (or a mixture thereof) may be used following a list of elements as shown in the following example where letters A-F represent the elements: “one or more elements selected from the group consisting of A, B, C, D, E, F, and a combination thereof.” The term, “a combination thereof” does not require that the combination include all of A, B, C, D, E, and F (although all of A, B, C, D, E, and F may be included). Rather, it indicates that a combination of any two or more of A, B, C, D, E, and F can be included. In other words, it is equivalent to the phrase “one or more elements selected from the group consisting of A, B, C, D, E, F, and a combination of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a combination thereof,” it indicates that that one or more of A, B, C, D, and F may be included, one or more of a salt of A, a salt of B, a salt of C, a salt of D, a salt of E, and a salt of F may be included, or a combination of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counter-ion. This list of counter-ions, however, is non-limiting.

Some of the compounds discussed throughout the disclosure may be in the form of a salt in the composition or added to the composition in the form of a salt (and dissociate in the composition). Thus, all compounds and amounts of compounds relate to both the salt form of the compound and to the disassociated form of the compound. In other words, even if the expression “a salt thereof” is not specifically or expressly stated with respect to ingredients that can form salts or are available as salts, it is understood that the salt form of the compound is included.

The expression “inclusive” for a range of concentrations means that the limits of the range are included in the defined interval.

The term “INCI” is an abbreviation of International Nomenclature of Cosmetic Ingredients, which is a system of names provided by the International Nomenclature Committee of the Personal Care Products Council to describe personal care ingredients.

All components and elements positively set forth in this disclosure can be negatively excluded from the claims. In other words, the compositions of the instant disclosure can be free or essentially free of all components and elements positively recited throughout the instant disclosure.

Some of the various categories of components identified may overlap. In such cases where overlap may exist and the composition includes both components (or the composition includes more than two components that overlap), an overlapping component or ingredient does not represent more than one component. For example, if a polyacrylate falls within the description of a thickening polymer and within the description of film forming polymer, the specifically recited polyacrylate is understood as being only the thickening polymer or only the film forming polymer. A specific polyacrylate cannot simultaneously be construed as both a thickening polymer and a film forming polymer, for example, when the claims set forth both a thickening polymer and a film forming polymer (even though the polyacrylate may function as both a thickening polymer and a film forming polymer). In other words, a single compound or ingredient cannot simultaneously serve as two different components of a claimed composition.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### Process for Manufacturing a Hair Cosmetic Composition

The hair care composition of the present invention can be manufactured from conventional cosmetic processes. An illustrative process comprises the following steps:
Step (1): in a vessel, adding from 30% to 70% of the water and the at least one organic acid, and mixing for about 10 minutes at room temperature;
Step (2): adding the at least one amino acid, mixing the ingredients until complete solubilization;
Step (3): adding the non-aqueous solvent comprising at least one compound with at least one hydroxyl-group, at least one fatty compound selected from vegetal oils, vegetal resins, vegetal waxes, and mixtures thereof and the at least one surfactant selected from cationic surfactants, nonionic surfactants, and mixtures thereof;
Step (4): heating the mixture until about 60° C. to about 75° C. for about 15 minutes.
Step (5): adding the remaining water into the mixture, and cooling down.
Step (6): adding additional ingredients (if present) cooling down until about 25° C., adjusting the pH (if necessary) with a pH adjuster.

### Embodiments

An example of a suitable hair cosmetic composition according to the present invention comprises:

In a preferred embodiment, the hair cosmetic composition of the present invention comprises:
(a) from about 0.5% to about 8.0% by weight of at least one organic acid selected from tartaric acid, lactic acid, glutaric acid, etidronic acid, salicylic acid, and a mixture thereof, preferably from tartaric acid, lactic acid, and mixtures thereof;
(b) from about 0.1% to about 20.0% by weight of non-aqueous solvent comprising at least one compound with at least one hydroxyl-group, said compound selected from polyols, C2 to Ce monoalcohols, and a mixture thereof;
(c) from about 0.1% to about 7.0% by weight of arginine;
(d) from about 0.1% to about 7.0% by weight of at least one fatty compound selected from vegetal oils, vegetal resins, vegetal waxes, and mixtures thereof;
(e) water; and
(f) from about 0.5% to about 7.0% by weight of a cationic surfactant selected from behentrimonium chloride, brassicamidopropyl dimethylamine, stearamidopropyl dimethylamine, and mixtures thereof and from about 6.0% to about 9.0% by weight of a nonionic surfactant selected from cetearyl alcohol, stearyl alcohol, and mixtures thereof.

Another preferred embodiment of the hair cosmetic composition of the present invention comprises:
(a) from about 0.5% to about 8.0% by weight of at least one organic acid selected from tartaric acid, lactic acid, glutaric acid, etidronic acid, salicylic acid, and a mixture thereof, preferably from tartaric acid, lactic acid, and mixtures thereof;
(b) from about 0.1% to about 20.0% by weight of a non-aqueous solvent comprising at least one compound with at least one hydroxyl-group, said compound selected from propylene glycol, C2 to Ce monoalcohols, glycerin, and a mixture thereof;
(c) from about 0.1% to about 7.0% by weight of arginine;
(d) from about 0.1% to about 7.0% by weight of Copaiba Officinalis (Balsam Copaiba) resin;
(e) water,
(f) from about 0.5% to about 7.0% by weight of a cationic surfactant selected from behentrimonium chloride, brassicamidopropyl dimethylamine, stearamidopropyl dimethylamine, and mixtures thereof and from about 6.0% to about 9.0% by weight of a nonionic surfactant selected from cetearyl alcohol, stearyl alcohol, and mixtures thereof; and
(g) from about 1.0% to about 5.0% by weight of at least one vegetal oil selected from coconut oil.

In some embodiments, the compositions of the present disclosure include from about 0.5% to about 1.2% by weight of glycine.

In some embodiments, the compositions of the present disclosure include from about 0.5% to about 5% by weight of esters selected from isopropyl esters (for example, isopropyl palmitate), dialkyl esters (for example, dicaprylyl carbonate), cetyl esters, and mixtures thereof.

In a further embodiment, the hair cosmetic composition of the present invention comprises:
(a) from about 0.5% to about 2.0% by weight of tartaric acid and lactic acid;
(b) from about 0.4% to about 3.5% by weight of propylene glycol, isopropyl alcohol and glycerin;
(c) from about 0.75% to about 2.5% by weight of arginine and glycine;
(d) from about 4.0% to about 6.0% by weight of Copaifera officinalis (balsam copaiba) resin and Cocos nucifera (coconut) oil;
(e) water;
(f) from about 5.0% to about 9.0% by weight of cetearyl alcohol and behentrimonium chloride;
(g) from about 1.0% to about 3.0% by weight of dicaprylyl carbonate and cetyl esters.

An additional suitable embodiment of the hair cosmetic composition of the present invention comprises:
(a) from about 2.0% to about 4.0% by weight of tartaric acid;
(b) from about 4.5% to about 9.5% by weight of propylene glycol, ethanol, isopropyl alcohol and cetearyl alcohol;
(c) from about 0.5% to about 2.0% by weight of glycine;
(d) from about 3.0% to about 5.0% by weight of Candelilla wax;
(e) water;
(f) from about 1.5% to about 2.5% by weight of behentrimonium chloride;
(g) from about 2.0% to about 5.5% by weight of isopropyl palmitate, dicaprylyl carbonate and cetyl esters.

### EXAMPLES

By way of non-limiting illustration, the invention will now be described with reference to the following examples.

### Examples 1 to 20

A suitable hair cosmetic composition according to the state-of-the art is as Examples 1 to 2, as follows:

| Function/Type | Ingredient | Ex. 1 | Ex. 2 |
|---|---|---|---|
| Amino Acid | Arginine | 1.0 | 1.0 |
| Organic Acid | Tartaric acid | 3.0 | 3.0 |
| Vegetal Resin | Copaifera officinalis | 0.17 | 0.17 |
| | (Balsam Copaiba) resin | | |
| Vegetal Oil | Cocos nucifera | 4.32 | 4.32 |
| | (coconut) oil | | |
| Non-Aqueous Solvent | Propylene glycol | 0.74 | 0.74 |
| Non-Aqueous Solvent | Ethanol | 0.55 | 0.55 |
| Non-Aqueous Solvent | Isopropyl alcohol | 0.54 | 0.54 |
| Surfactant | Cetearyl alcohol | 6.0 | 6.0 |
| Surfactant | Brassicamidopropyl | — | 1.5 |
| | dimethylamine | | |
| Alkanolamine | Triethanolamine | 1.2 | 1.2 |
| Surfactant | Behentrimonium chloride | 2.37 | 2.37 |
| Ester | Isopropyl palmitate | 1.5 | — |
| Ester | Dicaprylyl carbonate | 1.5 | 1.50 |
| Ester | Cetyl esters | 1.0 | 1.0 |
| Miscellaneous | one or more of fragrance, | </=2.0 | </=2.0 |
| Ingredients/Additives | preservatives, vitamins | | |
| Aqueous Solvent | Water | Q.S. | Q.S. |

Suitable hair cosmetic compositions according to the present invention are as Examples 3 to 10 as follows:

| | Ex. | Ex. | Ex. | Ex. | Ex. | Ex. | Ex. | Ex. |
|---|---|---|---|---|---|---|---|---|
| Ingredient | 3 | 4 | 5 | 6 | 7 | 8 | 9 | 10 |
| arginine | 1.0 | 1.0 | 1.0 | 1.0 | 1.0 | 1.0 | 1.0 | 1.0 |
| tartaric acid | 3.0 | 3.0 | 3.0 | 3.0 | 3.0 | 3.0 | 3.0 | 3.0 |
| Copaifera officinalis | 0.17 | 0.17 | 0.17 | 0.17 | 0.17 | 0.17 | 0.17 | 0.17 |
| (balsam copaiba) resin | | | | | | | | |
| Cocos nucifera | 4.32 | 4.32 | 4.32 | 4.32 | 4.32 | 4.32 | 4.32 | 4.32 |
| (coconut) oil | | | | | | | | |
| propylene glycol | 0.74 | 0.74 | 0.74 | 0.74 | 0.74 | 1.29 | 0.74 | 1.29 |
| Ethanol | 0.55 | 0.55 | 0.55 | 0.55 | 0.55 | 0.55 | 0.55 | — |
| Isopropyl alcohol | 0.54 | 0.54 | 0.54 | 0.54 | 0.54 | 0.54 | 0.54 | 0.54 |
| cetearyl alcohol | 6.0 | 6.0 | 6.0 | 6.0 | 6.0 | 6.0 | 6.0 | 6.0 |
| stearamidopropyl | — | — | 1.5 | 0.71 | 1.0 | — | — | — |
| dimethylamine | | | | | | | | |
| brassicamidopropyl | — | 1.5 | — | 1.24 | 1.86 | — | — | — |
| dimethylamine | | | | | | | | |
| triethanolamine | 1.2 | 1.2 | 1.2 | 1.2 | 1.2 | 1.5 | — | — |
| behentrimonium chloride | 2.37 | 2.37 | 2.37 | 2.37 | 2.37 | 2.37 | — | — |
| isopropyl palmitate | 1.5 | — | — | 1.25 | 0.7 | 1.5 | — | — |
| dicaprylyl carbonate | 1.5 | 1.5 | 1.5 | 0.5 | 2.42 | 1.5 | — | — |
| cetyl esters | 1.0 | 1.0 | 1.0 | 1.0 | 1.0 | 1.0 | 1.0 | 1.0 |
| one or more of fragrance, | </=2.0 | </=2.0 | </=2.0 | </=2.0 | </=2.0 | </=2.0 | </=2.0 | </=2.0 |
| preservatives, vitamins | | | | | | | | |
| water | Q.S. | Q.S. | Q.S. | Q.S. | Q.S. | Q.S. | Q.S. | Q.S. |

Additional hair cosmetic compositions according to the present invention are as Examples 11 to 14, as follows:

| Function/Type | Ingredient | Ex. 11 | Ex. 12 | Ex. 13 | Ex. 14 |
|---|---|---|---|---|---|
| Amino Acid | arginine | 1.0 | 1.0 | 1.0 | 1.0 |
| Organic Acid | tartaric acid | 1.17 | 1.17 | 1.17 | 1.17 |
| Organic Acid | lactic acid | 0.36 | 0.36 | 0.36 | 0.36 |
| Vegetal Resin | Copaifera officinalis | 0.17 | 0.17 | 0.17 | 0.17 |
| | (balsam copaiba) resin | | | | |
| Vegetal Oil | Cocos nucifera | 4.32 | 4.32 | 4.32 | 4.32 |
| | (coconut) oil | | | | |
| Non-Aqueous Solvent | propylene glycol | 1.29 | 1.29 | 1.29 | 1.29 |
| Non-Aqueous Solvent | isopropyl alcohol | — | — | 0.54 | 0.54 |
| Surfactant | cetearyl alcohol | 6.0 | 6.0 | 6.0 | 6.0 |
| Surfactant | stearyl alcohol | 2.0 | 3.0 | — | — |
| Surfactant | stearamidopropyl | 1.4 | 1.4 | — | — |
| | dimethylamine | | | | |
| Alkanolamine | triethanolamine | 0.45 | 0.5 | 0.7 | 0.7 |
| Surfactant | behentrimonium chloride | — | — | 2.37 | 2.37 |
| Ester | isopropyl palmitate | 1.5 | 1.5 | 1.5 | — |
| Ester | dicaprylyl carbonate | 1.5 | 1.5 | 1.5 | 1.0 |
| Ester | cetyl esters | 1.0 | 1.0 | 1.0 | 1.0 |
| Miscellaneous | one or more of fragrance, | </=2.0 | </=2.0 | </=2.0 | </=2.0 |
| ingredients/Additives | preservatives, vitamins | | | | |
| Aqueous Solvent | water | Q.S. | Q.S. | Q.S. | Q.S. |

Further hair cosmetic compositions according to the present invention are as Examples 15 to 19, as follows:

| Function/Type | Ingredient | Ex. 15 | Ex. 16 | Ex. 17 | Ex. 18 | Ex. 19 |
|---|---|---|---|---|---|---|
| Amino Acid | arginine | 1.0 | 1.0 | 1.0 | 1.0 | 1.0 |
| Amino Acid | glycine | — | 0.76 | 1.05 | 0.67 | 0.86 |
| Organic Acid | tartaric acid | 1.17 | 1.09 | 0.8 | 0.69 | 1.11 |
| Organic Acid | lactic acid | — | 0.43 | 0.79 | 0.99 | 0.17 |
| Vegetal resin | Copaifera | 0.17 | 0.17 | 0.17 | 0.17 | 0.17 |
| | officinalis | | | | | |
| | (balsam | | | | | |
| | copaiba) resin | | | | | |
| Vegetal Oil | Cocos nucifera | 4.32 | 4.02 | 0.57 | 0.79 | 4.16 |
| | (coconut) oil | | | | | |
| Non-Aqueous | isopropyl | 0.54 | 0.54 | 0.54 | 0.54 | 0.54 |
| Solvent | alcohol | | | | | |
| Non-Aqueous | propylene | 1.29 | 1.29 | 1.29 | 1.29 | 1.29 |
| Solvent | glycol | | | | | |
| Non-Aqueous | glycerin | — | 0.46 | 0.76 | 0.23 | 1.04 |
| Solvent | | | | | | |
| Surfactant | cetearyl | 6.0 | 6.0 | 6.0 | 6.0 | 6.0 |
| | alcohol | | | | | |
| Surfactant | behentrimonium | 2.37 | 2.37 | 2.37 | 2.37 | 2.37 |
| | chloride | | | | | |
| Ester | dicaprylyl | 1.0 | 1.0 | 1.0 | 1.0 | 1.0 |
| | carbonate | | | | | |
| Ester | cetyl esters | 1.0 | 1.0 | 1.0 | 1.0 | 1.0 |
| Miscellaneous | one or more of | </=2.0 | </=2.0 | </=2.0 | </=2.0 | </=2.0 |
| ingredients/ | fragrance, | | | | | |
| Additives | preservatives, | | | | | |
| | vitamins | | | | | |
| Alkanolamine | triethanolamine | 0.7 | 0.5 | 0.5 | 0.5 | 0.5 |
| Aqueous | water | Q.S. | Q.S. | Q.S. | Q.S. | Q.S. |
| Solvent | | | | | | |

The pH of the formulation examples of the invention ranged from about 3 to about 4. It was also found that the presence of the esters in the formulation examples of the invention and/or vegetal waxes improved the spreadability of the formulas during the application of the formulations onto hair.

### TESTS

### Test 1—Application and Long Lasting (Day after) Evaluation

A hair cosmetic composition according to Ex. 8 of the present invention was compared to a state-of-the-art composition comprising aqua, cetearyl alcohol, stearamidopropyl dimethylamine, dimethiconol, glycerin, parfum, lactic acid, paraffinum liquidum, methylparaben, petrolatum, DMDM hydantoin, disodium EDTA, cyclopentasiloxane, methoxypolyoxymethylene melamine, arginine, trimethylsiloxysilicate, benzyl benzoate, butylphenyl methylpropional, citronellol, geraniol, hexyl cinnamal, limonene, linalool.

In an in vivo, randomized half head study on the hair (curly type) of human volunteers, both hair cosmetic compositions were applied simultaneously to wet hair and evaluated with respect to various cosmetic attributes. The consumer/volunteer signals if there is a difference/no difference in the attributes' performance and if there is, it is described based on the intensity of the difference of the product (slight, medium, high or the same).

The evaluations were made at 4 time points: during application, while the hair was wet, when the hair became dry (without breaking the coating on the hair surface), and after breaking the coating on the hair (by running the fingers or a comb or a brush through the hair).

During the application of the hair cosmetic composition onto wet hair, the consumers/volunteers noticed a notable increase of penetration of the product into the hair (as evaluated according to the spreadability of the product from roots to ends of the hair fibers and the cosmetic feeling during the application). Better and lasting results were achieved.

The hair cosmetic composition according to Ex. 8 resulted in more definition of curls for all consumers/volunteers, as well as more manageability for the majority of the consumers/volunteers after breaking the coating on the hair.

Additionally, the volunteers stated that after a second application of the hair cosmetic compositions on the next day, the composition according to Ex. 8 resulted in more suppleness and curl definition on the hair.

To summarize, Formula Ex. 8 delivered significantly better and longer lasting curl definition to the hair, significantly improved the hair care and treatment routine by replacing several products in the routine and not needing to be reapplied, prolonging the time between washes, thereby resulting in less product being applied onto the hair. With less product being needed, the improved cosmetic effects were obtained such as the hair remaining light and natural.

### Test 2—Usage Experiments

In two separate studies, Hair cosmetic compositions according to Ex. 13 and Ex. 14 of the present invention were each compared to a state-of-the-art composition comprising water, cetearyl alcohol, stearamidopropyl dimethylamine, dimethiconol, glycerin, parfum, lactic acid, paraffinum liquidum, methylparaben, petrolatum, DMDM hydantoin, disodium EDTA, cyclopentasiloxane, methoxypolyoxymethylene melamine, arginine, trimethylsiloxysilicate, benzyl benzoate, butylphenyl methylpropional, citronellol, geraniol, hexyl cinnamal, limonene, linalool.

For each in vivo, randomized half head study on the hair (curly type) of human volunteers, six consumers/volunteers employed the hair cosmetic composition according to Ex. 14 as a leave-on refresher/styler on the hair after washing their hair with the hair cosmetic composition according to Ex. 14, without the need of touch-ups for up to three days.

The volunteers observed that the definition of the curls was maintained for increasingly longer intervals between washes, resulting in an extra day in the curly hair routine between applications for most of the consumers/volunteers. Particularly, the use of the hair cosmetic composition according to Ex. 14 delivered two more days without the need to re-apply the hair cosmetic composition of the invention. Thus, the hair cosmetic composition according to the invention did not have to be re-applied to the hair until the 3^{rd} day or the 4^{th} day after the first application.

In summary, Ex. 14 imparted lasting cosmetic effects and benefits on the hair, reaching up to the day after re-application at around 3 to 4 days from the first application. This provided the convenience of not needing to use multiple products on the hair or not needing to use multiple applications of a product on the hair on the same day or the day after the first application. It was also observed that a good balance between curl definition and a light feeling on the hair was achieved. These cosmetic effects and benefits were achieved by applying only a small amount of the hair cosmetic composition according to the disclosure on the hair.

In comparison, the consumers/volunteers stated that while using the mentioned state-of-the-art composition, there was a need to reapply the product multiple times a day because the curl definition didn't last long. Also, they observed a build-up of the product throughout multiple applications due to the necessity of multiple retouches, resulting in a shortening of the interval of washes for the curly hair routine.

Similar results were obtained when comparing the hair cosmetic composition according to Ex. 13 with the state-of-the-art composition.

During the first application, the application of the inventive composition according to Ex. 13 resulted in more individualized hair fibers. In addition, the inventive composition according to Ex. 13 had a trend to penetrate more on the hair and in Wet hair stage, the hair treated with the inventive composition according to Ex. 13 showed more curl definition and regularity, and less coating.

On the first day after the first application, the volunteers' hair that was treated with the inventive composition according to Ex. 13 exhibited more discipline and frizz control, more suppleness and better visual appearance.

On the day after the re-application of the test compositions on hair (i.e., last day of the study), it was observed that the hair cosmetic composition according to Ex. 13 provided more curl definition and curl regularity and more volume control to the hair when compared to the state-of-the-art composition.

### Test 3—Pure Comparative Evaluation

A hair cosmetic composition according to Ex. 12 of the present invention was compared to a state-of-the-art composition, comprising water, cetearyl alcohol, stearamidopropyl dimethylamine, dimethiconol, glycerin, parfum, lactic acid, paraffinum liquidum, methylparaben, petrolatum, DMDM hydantoin, disodium EDTA, cyclopentasiloxane, methoxypolyoxymethylene melamine, arginine, trimethylsiloxysilicate, benzyl benzoate, butylphenyl methylpropional, citronellol, geraniol, hexyl cinnamal, limonene, linalool.

The hair cosmetic composition according to Ex. 12 and the state-of-the-art composition were each tested in an in vivo, randomized half head study on the hair (curly type) of 6 human volunteers. The hair cosmetic composition according to Ex. 12 presented better performance in manageability attributes (more discipline and less volume and frizz) when compared to the results obtained by using the state-of-the-art composition, besides more curl regularity and better visual appearance in first day after the first application. In the second day after, the hair treated with the inventive composition maintained the attributes of curl regularity and better visual appearance, but showed more frizz, besides less cosmeticity.

### Test 4—In Vitro Tests on Hair Swatches

Formulas Exs. 14, 16, 17, 18, and 19 and the state-of-the-art composition were applied on virgin curly hair swatches (three 2.7-gram swatches per composition), and were assessed according to a curl definition scoring (from 0 to 10) at 24 hours after treating the hair (T24, 1^{st} time point), before breaking the coating on the hair (2^{nd} time point), and after breaking the coating (passing the fingers or a comb or a brush through the hair (third time point). The higher the score, the higher the degree of curl definition. The formula Ex. 14, 16, 17, 18, and 19 and the state-of-the-art composition had a curl score of from between about 7.5 to about 8.8 at the 1^{st} and 2^{nd} time points. The formula Ex. 14, 16, 17, 18, and 19 and the state-of-the-art composition had a curl score of from between about 7.4 to about 9 at the 3^{rd} time point. Formula Ex. 18 had a curl score of from between about 6 to about 6.5.

1. A hair cosmetic composition comprising:
(a) at least one organic acid;
(b) a non-aqueous solvent comprising at least one compound with at least one hydroxyl-group;
(c) at least one amino acid;
(d) at least one fatty compound selected from vegetal oils, vegetal resins, vegetal waxes, and mixtures thereof;
(e) water; and
(f) at least one surfactant selected from cationic surfactants, nonionic surfactants, and mixtures thereof.

2. The composition of claim 1, wherein the organic acid is selected from short chain α-hydroxy acids (AHAs), beta hydroxy acids, and a mixture thereof.

3. The composition of claim 1, wherein the at least one organic acid is selected from tartaric acid, lactic acid, glutaric acid, etidronic acid, salicylic acid, glycolic acid, citric acid, and mixtures thereof.

4. The composition of claim 1, wherein the at least one organic acid is in an amount of 0.1 to 25.0 wt. %, based on the total weight of the composition.

5. The composition of claim 1, wherein the non-aqueous solvent comprising at least one compound with at least one hydroxyl-group is selected from polyols, C₂ to C₆ monoalcohols, and mixtures thereof.

6. The composition of claim 5, wherein the polyol is selected from propylene glycol, dipropylene glycol, butylene glycol, pentylene glycol, hexylene glycol, glycerin, and mixtures thereof.

7. The composition of claim 1, wherein the non-aqueous solvent comprising at least one compound with at least one hydroxyl-group is in an amount of 0.1 to 20.0 wt. %, based on the total weight of the composition.

8. The composition of claim 1, wherein the at least one amino acid is selected from arginine, glycine, and mixtures thereof.

9. The composition of claim 8, wherein the at least one amino acid is arginine.

10. The composition of claim 1, wherein the at least one amino acid is in amount of 0.1 to 7.0 wt. %, based on the total weight of the composition.

11. The composition of claim 1, wherein the at least one fatty compound includes vegetal resins selected from Copaiba Officinalis (Balsam Copaiba) resin (oil resin or resin form), Candeia resin, white breu resin, and mixtures thereof.

12. The composition of claim 1, wherein the at least one fatty compound includes vegetal oils selected from coconut oil, soybean oil, and mixtures thereof.

13. The composition of claim 1, wherein the at least one fatty compound includes vegetal waxes selected from Candelilla wax, Carnauba wax, beeswax, and mixtures thereof.

14. The composition of claim 1, wherein the at least one fatty compound is in an amount of 0.1 to 14.0 wt. %, based on the total weight of the composition.

15. The composition of claim 1, wherein the at least one surfactant includes at least one cationic surfactant is a quaternary ammonium salt selected from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, and/or the at least one cationic surfactant is an amino amide cationizable surfactant selected from oleamidopropyl dimethylamine, linoleamidopropyl dimethylamine, isostearamidopropyl dimethylamine, stearamidopropyl dimethylamine, behenamidopropyl dimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, and mixtures thereof.

16. The composition of claim 15, wherein the at least one cationic surfactant is in an amount of 0.05 to 15.0 wt. %, based on the total weight of the composition.

17. The composition of claim 1, wherein the at least one surfactant includes at least one nonionic surfactant selected from at least one fatty alcohol selected from cetearyl alcohol, cetyl alcohol, stearyl alcohol, lauryl alcohol, myristyl alcohol, an alkoxylated alcohol, and mixtures thereof.

18. The composition of claim 17, wherein the at least one fatty alcohol is in a an amount of 1.0 to 12.0 wt. %, based on the total weight of the composition.

19. (canceled)

20. (canceled)

21. The composition of claim 1, wherein it further comprises cosmetically acceptable ingredients selected from perfume/fragrance, preserving agents, vitamins, vegetal extracts, pH-adjusters, and mixtures thereof.

22. A method for treating hair comprising contacting the hair with the hair cosmetic composition of claim 1.
